(19) United States
(12) Patent Application Publication
(10) Pub. No.: US 2024/0091763 A1
(43) Pub. Date: Mar. 21, 2024

(54) LIQUID HANDLING DEVICE AND LIQUID HANDLING METHOD

(71) Applicant: Enplas Corporation, Kawaguchi-shi (JP)

(72) Inventors: Koichi ONO, Kawaguchi (JP); Yasuhiro WATANABE, Kawaguchi (JP)

(21) Appl. No.: 18/283,183

(22) PCT Filed: Mar. 25, 2021
(86) PCT No.: PCT/JP2021/012639

§ 371 Date: Sep. 20, 2023

(51) Int. Cl. B01L 3/00

(52) U.S. Cl. CPC B01L 3/5027; B01L 2200/027

(57) ABSTRACT

The present invention pertains to a liquid handling device that comprises a laminate of a first member and a second member. This liquid handling device is provided with a reservoir that is a space for pooling a liquid and disposed between the first and second members, and a plurality of wells that are plurality of first recesses disposed on the surface of the first member facing the second member. The first and/or second members have flexibility. The first and second members are not bonded between the reservoir and the plurality of wells. When a part of the first or second member, said part corresponding to the reservoir, is pressed in the state where a liquid is pooled in the reservoir, the liquid in the reservoir moves between the first and second members toward the plurality of wells.

### TECHNICAL FIELD

The present invention relates to a liquid handling device and a liquid handling method.

### BACKGROUND ART

In recent years, a microwell chip has been used in order to conduct an analysis of a trace amount of substance such as a protein or a nucleic acid with high accuracy and at high speed. Advantageously, the microwell chip requires only a small amount of reagents and samples for the analysis, and are expected to be used in various applications such as clinical tests, food tests, and environmental tests (see, e.g., Patent Literature (hereinafter, referred to as “PTL” 1).

PTL 1 describes a microchannel device for use in polymerase chain reaction (PCR). The microchannel device described in PTL 1 includes an inlet for introducing a reaction solution, an outlet for discharging the reaction solution, a channel connecting the inlet and the outlet, and a plurality of cavities each of which is connected to the channel. In the microchannel device described in PTL 1, the reaction solution is introduced into a plurality of cavities from the inlet. Then, the PCR is performed by thermal cycling of the reaction solution in the cavities.

### CITATION LIST

### Patent Literature

### PTL 1

Japanese Patent Application Laid-Open No. 2018-117601

### SUMMARY OF INVENTION

### Technical Problem

Generally, in a PCR test or the like, a plurality of reaction solutions are handled at a time to improve the test efficiency. In the microchannel device described in PTL 1, it is conceivable to increase the number of cavities in order to improve the test efficiency of the PCR test. In the microchannel device described in PTL 1, when the number of cavities is increased, it is necessary to shorten the channel between the cavities to increase the arrangement density of the cavities. However, in this case, it is conceivable that the manufacturing cost of the microchannel device increases.

An object of the present invention is to provide a liquid handling device capable of being inexpensively manufactured even when the arrangement density of the wells is increased. Another object of the present invention is to provide a liquid handling method using the liquid handling device.

### Solution to Problem

A liquid handling device according to the present invention is a liquid handling device including a laminate of a first member and a second member, the liquid handling device including: a reservoir that is a space disposed between the first member and the second member and configured to store a liquid; and a plurality of wells that are a plurality of first recesses disposed in a surface of the first member facing the second member, in which at least one of the first member and the second member is flexible, and the first member and the second member are not joined to each other between the reservoir and the plurality of wells, and, when a portion of the first member or the second member corresponding to the reservoir is pressed in a state in which the liquid is stored in the reservoir, the liquid in the reservoir moves between the first member and the second member toward the plurality of wells.

A liquid handling method of the present invention includes: preparing a liquid handling device of the present invention; introducing a liquid into the reservoir; and pressing a portion of the first member or the second member corresponding to the reservoir so as to move the liquid in the reservoir toward the plurality of wells between the first member and the second member.

### Advantageous Effects of Invention

According to the present invention, it is possible to provide a liquid handling device that can be inexpensively manufactured even when the arrangement density of wells is increased.

### BRIEF DESCRIPTION OF DRAWINGS

FIG. 1 is a perspective view of a liquid handling device according to an embodiment of the present invention;

FIGS. 2A to 2C illustrate a configuration of a liquid handling device according to an embodiment of the present invention;

FIGS. 3A and 3B are perspective views of a first member;

FIG. 4 is a perspective view of a second member;

FIGS. 5A to 5D are sectional schematic views for explaining a liquid handling method according to an embodiment of the present invention;

FIGS. 6A to 6C are other sectional schematic views for explaining the liquid handling method;

FIGS. 7A to 7C are schematic plan views for explaining the liquid handling method;

FIG. 8 is a schematic diagram illustrating a configuration of a PCR device; and

FIGS. 9A to 9C are schematic partial sectional views for liquid handling methods according to variations of the present embodiment.

### DESCRIPTION OF EMBODIMENTS

Hereinafter, an embodiment of the present invention will be described in detail with reference to the drawings.

In the present embodiment, liquid handling device 100 capable of performing PCR in a plurality of wells 180 will be described.

(Liquid Handling Device)

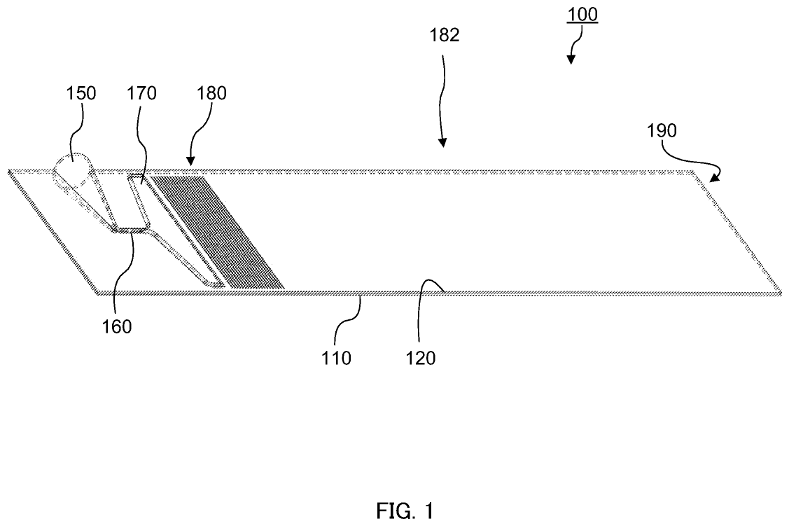

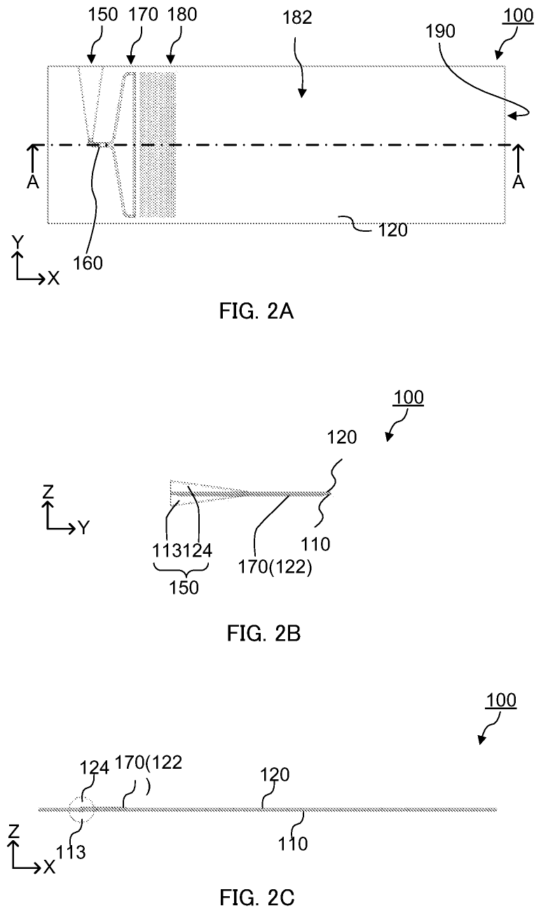

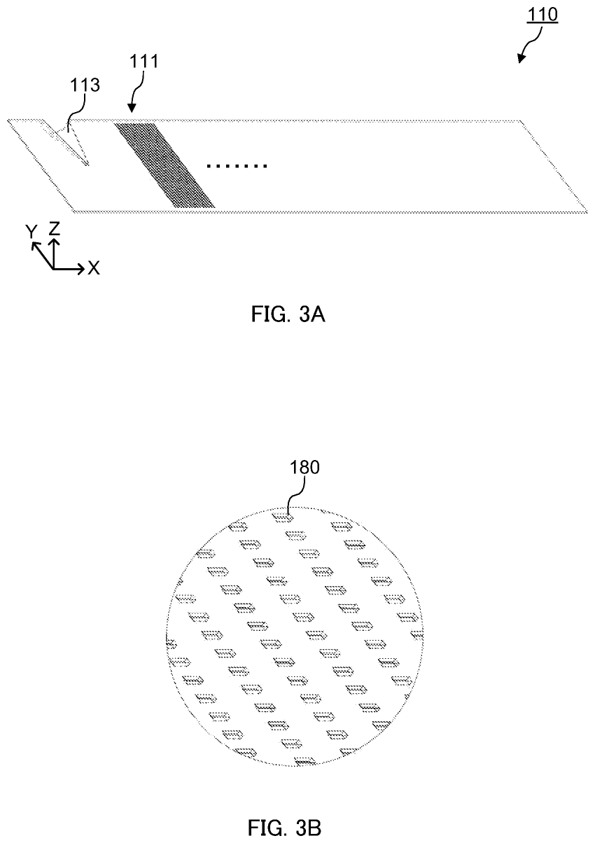

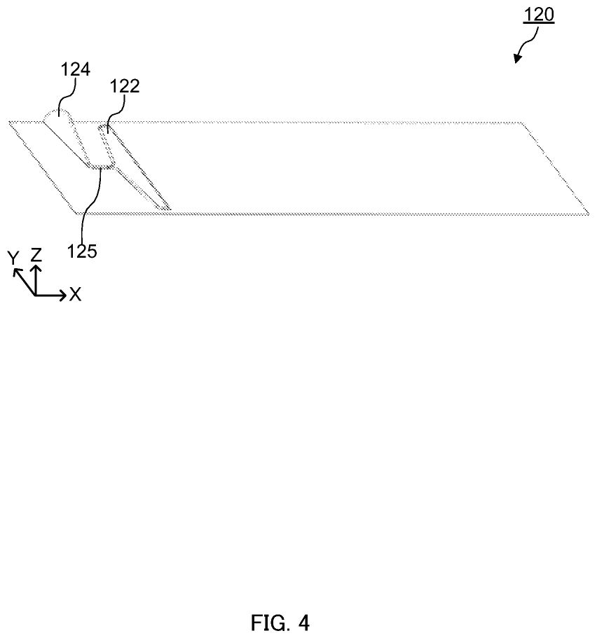

FIG. 1 is a perspective view of liquid handling device 100 according to an embodiment of the present invention. FIG. 2A is a plan view of liquid handling device 100 according to the embodiment of the present invention. FIG. 2B is a left side view of liquid handling device 100. FIG. 2C is a sectional view taken along line A-A illustrated in FIG. 2A. FIG. 3A is a perspective view of first member 110. FIG. 3B is a partially enlarged perspective view of a region of first member 110 in which a plurality of first recesses 111 are formed. FIG. 4 is a perspective view of second member 120. For ease of illustration, illustration of first recesses 111 is omitted in the right-side region of FIG. 3A. In the following description, a direction along the long sides of liquid handling device 100 as seen in a plan view is referred to as an “X direction,” a direction along the short sides of liquid handling device 100 as seen in a plan view is referred to as a “Y direction,” and a direction orthogonal to the X direction and the Y direction is referred to as a “Z direction.”

As illustrated in FIGS. 1 and 2A to 2C, liquid handling device 100 includes a laminate of first member 110 and second member 120. First member 110 and second member 120 are partially joined to each other. At least one of first member 110 and second member 120 is flexible. In the present embodiment, both of first member 110 and second member 120 are flexible films. Herein, the “films” mean thin films that are flexible and whose thickness is less than 0.3 mm.

As illustrated in FIG. 3A, first member 110 has a plurality of first recesses 111 and third recess 113 in the present embodiment. The thickness of first member 110 is not particularly limited as long as it is larger than the depth of the plurality of first recesses 111 and allows first member 110 to be flexible and to deform when first member 110 is pressed. The thickness of first member 110 is, for example, within the range of 0.02 to 0.3 mm. The material of first member 110 may be appropriately selected from known resins. Examples of the material of first member 110 include polyethylene terephthalate, polycarbonate, polymethyl methacrylate, polyvinyl chloride, polypropylene, polyether, polyethylene, polystyrene, cycloolefin-based resins, silicone resins, and elastomers.

The plurality of first recesses 111 form a plurality of wells 180 when first member 110 is partially joined to second member 120. The shape of each of first recesses 111 is not particularly limited. As illustrated in FIG. 3B, the shape of each of first recesses 111 is a shape of a trapezoidal column that opens in the upper surface in the present embodiment.

Third recess 113 is a recess that forms a part of introduction portion 150 when first member 110 is partially joined to second member 120. The shape of third recess 113 is not particularly limited as long as introduction of a liquid is possible. In the present embodiment, third recess 113 has a shape of a cone bisected along the height direction.

As illustrated in FIG. 4, second member 120 has second recess 122 and fourth recess 124 in the present embodiment. The thickness of second member 120 is not particularly limited as long as it allows second member 120 to be flexible and to deform when the second member is pressed. In the present embodiment, the thickness of second member 120 is, for example, within the range of 0.02 to 0.3 mm. The material of second member 120 may be appropriately selected from known resins. Examples of materials for second member 120 include polyethylene terephthalate, polycarbonate, polymethyl methacrylate, polyvinyl chloride, polypropylene, polyether, polyethylene, polystyrene, cycloolefin-based resins, silicone resins, and elastomers. Second member 120 may be formed of the same material as first member 110 or may be formed of a different material from that of the first member. In the present embodiment, the material of second member 120 is the same as the material of first member 110. Second recess 122 and fourth recess 124 are connected by connection groove 125.

Second recess 122 is a recess that forms a part of reservoir 170 when first member 110 is partially joined to second member 120. Connection groove 125 is connected to second recess 122. The shape of second recess 122 is not particularly limited as long as the liquid can be stored therein. In the present embodiment, second recess 122 is formed such that its width (Y direction) gradually increases as the distance from connection groove 125 increases. The width of the end portion of second recess 122 opposite to connection groove 125 is preferably the same as or longer than the width of the plurality of first recesses 111 disposed.

Fourth recess 124 is a recess that forms a part of introduction portion 150 when first member 110 is partially joined to second member 120. The shape of fourth recess 124 is not particularly limited as long as introduction of a liquid is possible. In the present embodiment, fourth recess 124 has a shape of a cone bisected along the height direction. In the present embodiment, connection groove 125 is disposed in the tip end of fourth recess 124.

Connection groove 125 is a groove that forms connection channel 160 when first member 110 is partially joined to second member 120. Second recess 122 is connected to one end of connection groove 125, and fourth recess 124 is connected to the other end of connection groove 125.

As described above, first member 110 and second member 120 are partially joined to each other. The joining method for joining first member 110 and second member 120 is not particularly limited. Examples of the joining method for joining first member 110 and second member 120 include thermal welding, laser welding, and joining with an adhesive or the like. In the present embodiment, first member 110 and second member 120 are joined to each other by thermal welding. In the present embodiment, first member 110 and second member 120 are not joined to each other in well formation region 182. Further, first member 110 and second member 120 are not joined to each other between reservoir 170 and well formation region 182. Further, first member 110 and second member 120 are not joined to each other at the downstream end portion in a direction along the direction in which the liquid flows (X direction). First member 110 and second member 120 are joined to each other at positions other than the regions described above. First member 110 and second member 120 are joined to each other at opposite end portions of well formation region 182 in the Y direction.

Liquid handling device 100 includes reservoir 170 and wells 180. Liquid handling device 100 may further include introduction portion 150, connection channel 160, and discharge portion 190. In the present embodiment, liquid handling device 100 includes introduction portion 150, connection channel 160, reservoir 170, wells 180, and discharge portion 190.

The shape of liquid handling device 100 as seen in plan view is not particularly limited. In the present embodiment, the shape of liquid handling device 100 as seen in plan view is a rectangle surrounded by two opposed long sides and two opposed short sides.

Introduction portion 150 is used for introducing a liquid into reservoir 170. One end of introduction portion 150 is open to the outside, and the other end is connected to connection channel 160. Introduction portion 150 is formed by joining first member 110 and second member 120 to each other. In the present embodiment, introduction portion 150 includes third recess 113 disposed in first member 110 and fourth recess 124 disposed in second member 120. In the present embodiment, the shape of introduction portion 150 is a conical shape corresponding to the shape of a pipette tip. Further, the size of introduction portion 150 is not particularly limited, and may be appropriately set according to the application. In the present embodiment, the size of introduction portion 150 corresponds to the size of the pipette tip. The opening portion of introduction portion 150 may be disposed at a position corresponding to the long sides or may be disposed at a position corresponding to the short sides of liquid handling device 100 as seen in plan view. In the present embodiment, the opening portion of introduction portion 150 is disposed at a position corresponding to the long sides of liquid handling device 100 as seen in plan view. Further, the central axis of the cone is disposed to be parallel to the short sides of the rectangle.

Connection channel 160 connects introduction portion 150 to reservoir 170. The length, the cross-sectional shape, and the like of connection channel 160 are not particularly limited as long as the liquid introduced into introduction portion 150 can be sent to reservoir 170, and are appropriately set.

Reservoir 170 stores the liquid introduced from introduction portion 150. Reservoir 170 is formed by partially joining first member 110 and second member 120 to each other. More specifically, while a region of second member 120 around second recess 122 and a region of first member 110 corresponding to this region of the second member are joined to each other, first member 110 and second member 120 are not joined to each other between reservoir 170 and the plurality of wells 180. Accordingly, the liquid stored in reservoir 170 flows into wells 180. In the present embodiment, reservoir 170 includes first recesses 111 disposed in first member 110 and a flat portion of second member 120 disposed to face first recesses 111. The volume of reservoir 170 is not particularly limited, and is appropriately set according to the amount of a reaction solution.

Each of the plurality of wells 180 stores the liquid introduced from reservoir 170. The plurality of wells 180 are disposed in well formation region 182. Well formation region 182 is disposed in a region (right-side region) of liquid handling device 100 as seen in plan view, which region is opposite to introduction portion 150. The region of first member 110 around first recesses 111 and the region of second member 120 corresponding to this region are not joined to each other. In addition, the opposite end portions of first member 110 and second member 120 in the direction orthogonal to the direction along which the liquid flows are joined to each other. In the present embodiment, wells 180 are formed by first recesses 111 and the flat portion of second member 120 disposed in the region facing first recesses 111. The volume of each of wells 180 is, for example, in the range of a few μL to a few hundred μL. The number of wells 180 is not particularly limited as long as there are a plurality of wells, and is appropriately set. The arranging method for arranging the plurality of wells 180 is not particularly limited. The plurality of wells 180 may be arranged on grid points of a rectangular grid, or may be arranged in a staggered manner.

Discharge portion 190 is an opening portion for discharging the liquid that has not been introduced into wells 180. Discharge portion 190 is formed by not joining first member 110 and second member 120 partially to each other. More specifically, in the present embodiment, discharge portion 190 is an opening portion formed by first member 110 and second member 120 and disposed on the downstream side of the region where wells 180 are disposed. The discharge portion is formed by joining the opposite end portions in the direction (Y direction) orthogonal to the direction (X direction) in which the liquid flows between first member 110 and second member 120 and by not joining the downstream end portions in the direction (X direction) in which the liquid flows.

(Liquid Handling Method)

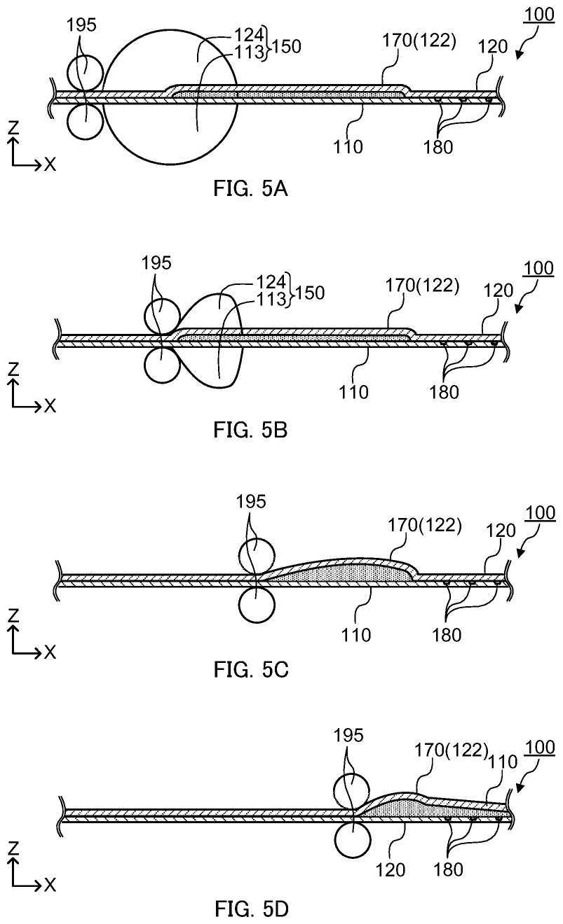

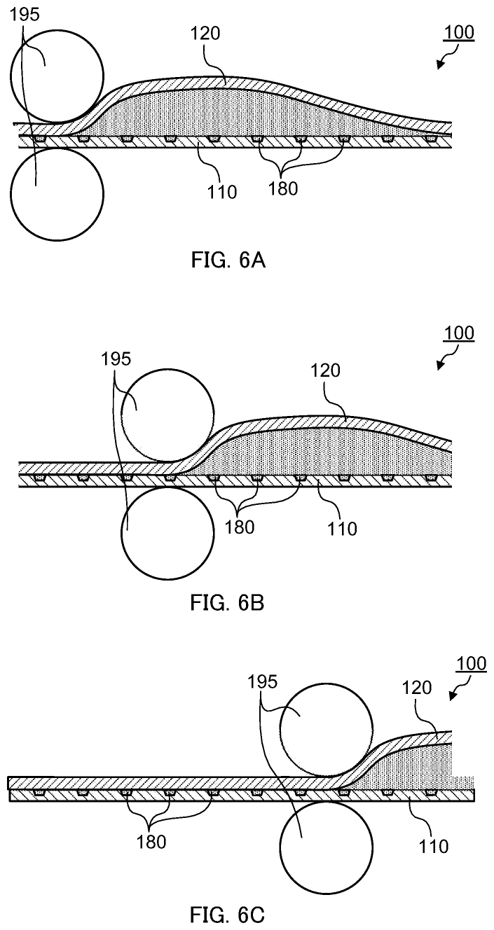

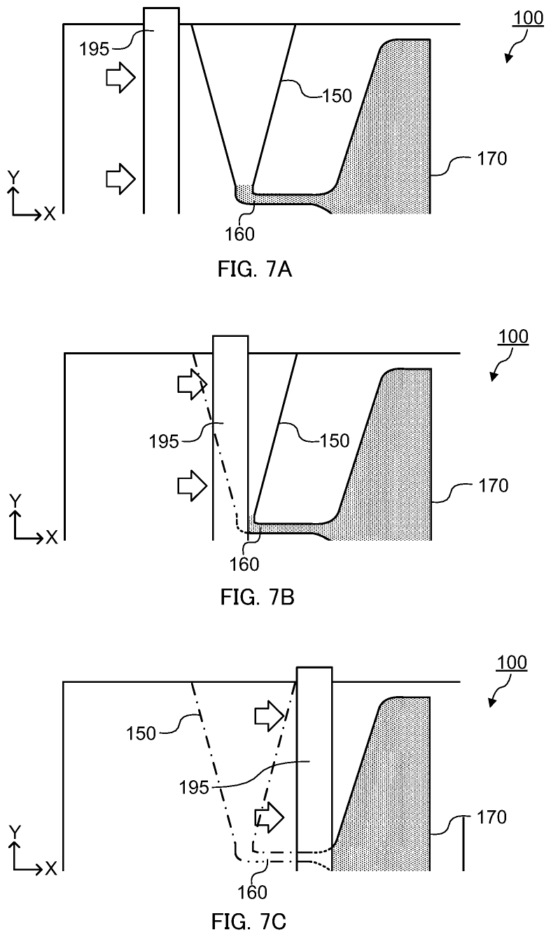

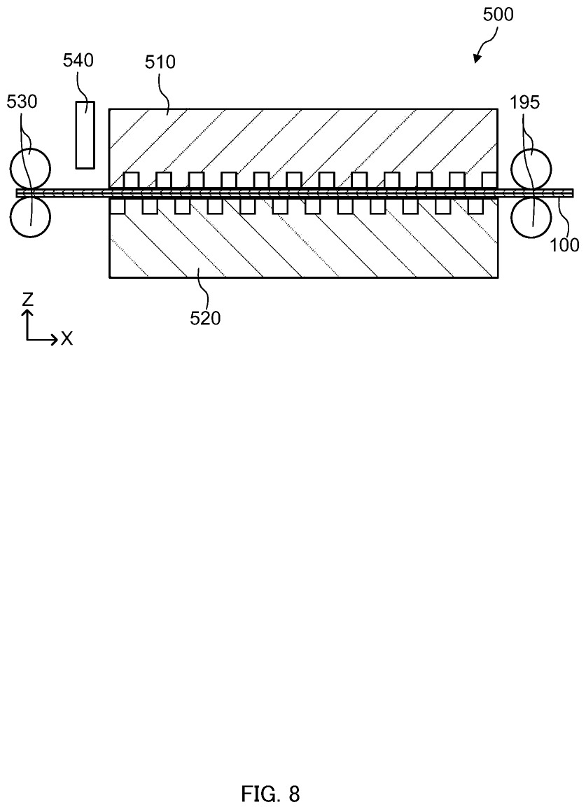

In the following, a liquid handling method according to the present embodiment will be described. FIGS. 5A to 5D and 6A to 6C are schematic sectional views for explaining the liquid handling method. FIGS. 7A to 7C are schematic plan views for explaining the liquid handling method. FIG. 8 is a schematic diagram illustrating a configuration of PCR device 500.

The liquid handling method according to the present embodiment includes a step of preparing above-described liquid handling device 100, a step of introducing a liquid into reservoir 170, and a step of moving the liquid to wells 180.

In the step of preparing liquid handling device 100, liquid handling device 100 having the above-described configuration may be manufactured, or a commercially available product may be purchased.

In the step of introducing the liquid into reservoir 170, the liquid is introduced from introduction portion 150 into reservoir 170. In the present embodiment, introduction portion 150 has a shape substantially complementary to the shape of the pipette tip, and the liquid is introduced in a state in which the pipette tip of the pipette is inserted into introduction portion 150. The introduced liquid is stored in reservoir 170 via connection channel 160. It is preferable that connection channel 160 be filled with the liquid up to the upstream end portion. In addition, the liquid stored in reservoir 170 remains in reservoir 170 without moving toward wells 180.

In the step of moving the liquid, a portion of first member 110 or second member 120 corresponding to reservoir 170 are pressed to move the liquid in reservoir 170 toward the plurality of wells 180 between first member 110 and second member 120. The method of pressing the portion of first member 110 or second member 120 corresponding to reservoir 170 is not particularly limited as long as it moves the liquid in reservoir 170 toward wells 180. In the present embodiment, the liquid is moved by using a pair of first rollers 195 and 195.

Specifically, as illustrated in FIGS. 5A and 7A, liquid handling device 100 is sandwiched between a pair of heated first rollers 195 and 195 at an end portion on the side where introduction portion 150 is disposed. Next, the pair of first rollers 195 and 195 are rotated and moved from the region of liquid handling device 100 on the introduction portion 150 side toward the region on the wells 180 side (in the X direction).

As illustrated in FIGS. 5B, 5C, 7B, and 7C, the pair of first rollers 195 and 195 that have reached the portion of first member 110 or second member 120 corresponding to introduction portion 150 move while continuously thermally welding the inner surface of third recess 113 and the inner surface of fourth recess 124. Thus, first member 110 and second member 120 after pressed at the portion of first member 110 or second member 120 corresponding to introduction portion 150 are joined to each other. Therefore, in the liquid handling method according to the present embodiment, it is possible to prevent air bubbles from entering from introduction portion 150.

As illustrated in FIG. 5D, when the pair of first rollers 195 and 195 continuously press the portion of first member 110 or second member 120 corresponding to reservoir 170, the liquid passes through the opening edge of second recess 122 and moves toward wells 180.

As illustrated in FIGS. 6A to 6C, the liquid that has passed through the opening edge of second recess 122 is introduced into wells 180. At this time, in the present embodiment, the pair of first rollers 195 and 195 move in the X direction while thermally welding first member 110 and second member 120. Therefore, an opening portion of well 180 which the pair of first rollers 195 and 195 have passed is sealed. As described above, since the opening portions of the plurality of wells 180 are continuously sealed by passing the pair of first rollers 195 and 195, the arrangement density of wells 180 can be increased and contamination can be prevented.

In the present embodiment, the pair of first rollers 195 and 195 move while thermally welding first member 110 and second member 120, but thermal welding is not necessary. For example, if the liquid does not move from one well 180 to another after the pair of first rollers 195 and 195 passes, the pair of first rollers 195 and 195 need not thermally weld first member 110 and second member 120. In the present embodiment, the pair of first rollers 195 and 195 is a part of PCR device 500 described later.

Then, PCR is performed, for example, by repeating thermal cycling.

As illustrated in FIG. 8, PCR device 500 that performs PCR includes first heating section 510, second heating section 520, a pair of first rollers 195, a pair of second rollers 530, and sensor 540. First heating section 510 heats the liquid to, for example, 95° C. First heating section 510 includes a plurality of first heating regions 512. Second heating section 520 heats the liquid to, for example, 65° C. Second heating section 520 includes a plurality of second heating regions 522. First heating section 510 and second heating section 520 are disposed to face each other, and first heating region 12 and second heating region 522 are disposed in a staggered manner in a direction in which liquid handling device 100 containing the liquid in each well 180 moves. Thus, by moving liquid handling device 100, it is possible to repeatedly perform heating and cooling. The pair of first rollers 195 is disposed on the upstream side in the moving direction of liquid handling device 100. The pair of second rollers 530 is disposed on the downstream side in the moving direction of liquid handling device 100. The pair of first rollers 195 and the pair of second rollers 530 cooperate to move liquid handling device 100 inside PCR device 500. Since the pair of first rollers 195 is disposed on the upstream side of liquid handling device 100 of PCR device 500, PCR can be continuously performed while the liquid is introduced into wells 180. Sensor 540 detects, for example, fluorescent light generated from the liquid after PCR. A detection value such as fluorescent light is detected from the liquid after PCR using sensor 540.

(Effects)

As described above, in liquid handling device 100 according to the present embodiment, the liquid in reservoir 170 is introduced into the plurality of wells 180 by pressing reservoir 170. It is thus not necessary to form a channel from reservoir 170 to the plurality of wells 180. Therefore, even when the arrangement density of wells 180 is increased, it can be manufactured at low cost. It is also possible to prevent contamination between wells 114.

(Variations)

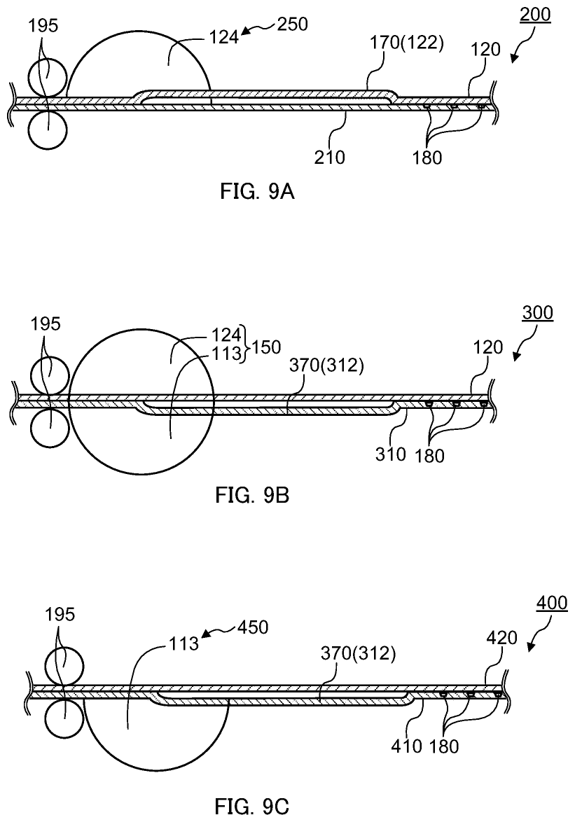

Next, liquid handling devices 200, 300, and 400 according to variations of the present embodiment will be described. FIGS. 9A to 9C are partial sectional schematic views of liquid handling devices 200, 300, and 400 according to the variations of the present embodiment. FIG. 9A is a schematic sectional view of liquid handling device 200 according to Variation 1 of the present embodiment. FIG. 9B is a schematic sectional view of liquid handling device 300 according to Variation 2 of the present embodiment. FIG. 9C is a schematic sectional view of liquid handling device 400 according to Variation 3 of the present embodiment.

As illustrated in FIG. 9A, introduction portion 250 may include fourth recess 124 and a flat portion of first member 210 disposed in a region facing fourth recess 124. Thus, the thickness of first member 210 can be increased, and first member 210 can be easily manufactured. Further, although not particularly illustrated in the figures, introduction portion 250 may include third recess 113 and a flat portion of second member 120 disposed in a region facing third recess 113. In this case, second member 210 may not be flexible.

As illustrated in FIG. 9B, in liquid handling device 300, second recess 312 may be disposed in first member 310.

Further, as illustrated in FIG. 9C, in liquid handling device 400, second recess 312 may be disposed in first member 410, and introduction portion 450 may include third recess 113 and a flat portion of second member 420 disposed in a region facing third recess 113. In this case, second member 420 does not have to be flexible. Accordingly, since it is not necessary to form recesses in second member 420, the manufacturing cost can be reduced.

(Effects)

As described above, liquid handling devices 200, 300, and 400 according to the variations have the same effects as those of liquid handling device 100 according to the embodiment. In addition, liquid handling devices 200 and 400 can further reduce the manufacturing cost.

### INDUSTRIAL APPLICABILITY

The liquid handling device of the present invention is useful in various applications such as clinical testing, food testing, and environmental testing.

### REFERENCE SIGNS LIST

100, 200, 300, 400 Liquid handling device
110, 210, 310, 410 First member
111 First recess
113 Third recess
114 Well
120, 420 Second member
122, 212, 312 Second recess
124 Fourth recess
125 Connection groove
150, 250, 450 Introduction portion
160 Connection channel
170 Reservoir
180 Well
182 Well formation region
190 Discharge portion
195 Roller
500 PCR device
510 First heating section
520 Second heating section
530 Second roller
540 Sensor

1. A liquid handling device including a laminate of a first member and a second member, the liquid handling device comprising:
a reservoir that is a space disposed between the first member and the second member and configured to store a liquid; and
a plurality of wells that are a plurality of first recesses disposed in a surface of the first member facing the second member, wherein
at least one of the first member and the second member is flexible,
the first member and the second member are not joined to each other between the reservoir and the plurality of wells, and
when a portion of the first member or the second member corresponding to the reservoir is pressed in a state in which the liquid is stored in the reservoir, the liquid in the reservoir moves between the first member and the second member toward the plurality of wells.

2. The liquid handling device according to claim 1, further comprising:
an introduction portion connected to the reservoir and configured to introduce a liquid into the reservoir; and
a discharge portion disposed on a side of the plurality of wells opposite to the reservoir and configured to discharge the liquid having moved between the first member and the second member.

3. The liquid handling device according to claim 1, wherein
the reservoir comprises:
a second recess disposed in the second member, and
a flat portion of the first member disposed in a region facing the second recess.

4. The liquid handling device according to claim 2, wherein
the introduction portion comprises:
a third recess disposed in the first member, and
a fourth recess disposed in the second member in a region facing the third recess.

5. The liquid handling device according to claim 1, wherein
the first member or the second member is a film.

6. A liquid handling method, comprising:
preparing a liquid handling device according to claim 1;
introducing a liquid into the reservoir;
pressing a portion of the first member or the second member corresponding to the reservoir so as to move the liquid in the reservoir toward the plurality of wells between the first member and the second member.

7. The liquid handling method according to claim 6, wherein
the first member and the second member after the pressing of the portion of the first member or the second member corresponding to the reservoir are joined to each other.
